(19) United States
(12) Patent Application Publication
(10) Pub. No.: US 2023/0165033 A1
(43) Pub. Date: May 25, 2023

(54) MXENE TRANSPARENT CONDUCTING LAYERS FOR DIGITAL DISPLAY AND METHOD THEREOF

(71) Applicant: DREXEL UNIVERSITY, Philadelphia, PA (US)

(72) Inventors: Tae-woo LEE, Seocho-Gu (KR); Soyeong AHN, Yeongtong-gu (KR); Kathleen Ann MALESKI, Mount Airy, MD (US); Yury GOGOTSI, Ivyland, PA (US)

(21) Appl. No.: 17/995,184

(22) PCT Filed: Apr. 2, 2021
(86) PCT No.: PCT/US2021/025497

§ 371 Date: Sep. 30, 2022

Related U.S. Application Data
(60) Provisional application No. 63/004,069, filed on Apr. 2, 2020.

(51) Int. Cl. H10K 50/81; G02F 1/155; H10K 50/17; G01N 21/552; C01B 32/921; C01B 32/907

(52) U.S. Cl. CPC H10K 50/81; G02F 1/155; H10K 50/17; G01N 21/553; C01B 32/921; C01B 32/907; G02F 2202/16; H10K 2102/10

(57) ABSTRACT

Provided are MXene-containing electrodes, display devices, electrochromic devices, and other optoelectronic devices, which devices can include transparent and/or colored MXene materials. In particular, MXenes can be used as transparent conducting electrodes based on their comparatively high electrical conductivity and high work function. An electrode, comprising: a substrate; a portion of MXene material disposed on the substrate; a hole-injection material disposed on the MXene material; an organic layer in electronic communication with the hole-injection material; and a conductor material in electronic communication with the hole-injection material.

### RELATED APPLICATIONS

The present application claims priority to and the benefit of U.S. patent application No. 63/004,069, “MXene Transparent Conducting Layers For Digital Display And Method Thereof” (filed Apr. 2, 2020), the entirety of which application is incorporated herein by reference for any and all purposes.

### TECHNICAL FIELD

The present disclosure relates to the field of MXene materials and their use in display applications.

### BACKGROUND

Indium tin oxide (ITO) has been widely-used as a transparent conducting electrode (TCE) for optoelectronic devices, but it has a number of disadvantages such as brittleness, increased cost, and diffusion of metal species into devices. Several other flexible conducting materials including carbon nanotubes, graphene and conducting polymers have been considered as an alternative solution, providing flexibility to the TCE and their usages were intensively explored for flexible optoelectronics.

Even though there has been substantial progress in flexible optoelectronic applications, still a number of technical issues remain. To achieve high quality graphene, a high-temperature chemical vapor deposition (CVD) growth at ˜1000° C., and additional charge transfer doping are usually required to improve the electrical conductivity of intrinsic graphene sheets.

The graphene doped with volatile chemicals has electrical instability issues, complicating fabrication of devices. Conducting polymers (e.g., poly(3,4-ethylenedioxythiophene) polystyrene sulfonate (PEDOT:PSS)) have been limited by the relatively low electrical conductivity (<1000 S/cm)^{[19-21].} Furthermore, the work function (WF) of conducting materials is usually less than 4.8 eV, which also acts as a critical limiting factor for organics, quantum dots, and perovskite light-emitting diodes (LEDs) due to a large energy barrier formation for hole injection. Accordingly, there is a long-felt need for improved such electrodes.

### SUMMARY

In meeting the described needs, the present disclosure provides an electrode, comprising: a substrate; a portion of MXene material disposed on the substrate; a hole-injection material disposed on the MXene material; an organic layer in electronic communication with the hole-injection material; and a conductor material in electronic communication with the hole-injection material.

Also provided are methods, comprising fabricating an electrode according to the present disclosure

Further provided are methods, comprising the use of an electrode according to the present disclosure.

Additionally provided are display devices, comprising an electrode according to the present disclosure.

Also disclosed is a method, comprising: exposing a plurality of MXene samples to illumination; collecting optical spectra from the plurality of MXene samples; and classifying at least one of the plurality of MXene samples based on an optical spectrum of that at least one of the plurality of MXene samples.

Further provided is a photothermal therapy method, comprising: exposing a MXene material sample disposed on or within a subject to near-infrared radiation, the exposing effecting localized heating of a tissue of the subject.

Also provided is an electrochromic device, comprising a portion of MXene material; and an electrical current source, the electrical current source in electronic communication with the MXene material, the electrical current source configured to effect application of an electrical current sufficient to effect a change in color, transparency, or both in the MXene material.

Additionally provided is a sensor, comprising: a MXene portion in electronic communication with a detector configured to detect a signal from the MXene portion, the MXene portion being essentially transparent to visible light.

Also disclosed is a sensor, comprising: a MXene portion in electronic communication with a detector configured to detect a signal from the MXene portion, the MXene portion being colored.

### BRIEF DESCRIPTION OF THE DRAWINGS

FIG. 1 provides a schematic illustration of a) MXene film formation using Ti₃C₂ MXene solution by spin-coating on ozone-treated substrate. b) scanning electron microscopy (SEM) and c) atomic force microscopy (AFM) images of prepared Ti₃C₂ film. d) characterization of optoelectronic properties. From left to right: Transmittance T [%] versus sheet resistance R_{S} [Ω/sq]); slope of T^{0.5}−1 versus thickness yields optical conductivity [S·cm⁻¹]; slope of R_{S} versus thickness [cm] or inverse thickness [cm⁻¹) yields DC conductivity [S·cm⁻¹]. e) R_{S} normalized to initial value Rso of ITO and MXene after bending up to 5000 times.

FIG. 2 provides a) Contact potential difference (CPD) of ITO and Ti₃C₂ MXene electrodes measured by Kelvin probe. b) Schematic illustration of MXene sheets and surface functional groups (—OH, —O and —F) on them. c) X-ray photoelectron spectroscopy (XPS) O 1 s, F 1 s spectra and d) CPD of MXene films with low-temperature vacuum annealing at room temperature (RT) (˜23° C.), 100° C., and 200° C., or without vacuum annealing, with ITO as a comparison.

FIG. 3 provides a) Delamination of Ti₃C₂ MXene during spin-coating of PEDOT:PSS and b) SEM images of delaminated MXene film. c) Chemical structures of constituents (PEDOT:PSS, aniline and perfluorinated polymer) of n-GraHIL, and schematic of n-GraHIL formation on Ti₃C₂ MXene film. d) MXene uniformly covered by n-GraHIL solution without delamination, e) and transmittance of MXene films without and with n-GraHIL. XPS depth profiles of f) Ti₃C₂ MXene, g) MXene/PEDOT:PSS and h) MXene/n-GraHIL.

FIG. 4 provides a) Schematic illustrating the hole-only device design b) Current density versus voltage characteristics and c) calculated hole injection efficiency (1) of hole-only device with ITO/PEDOT:PSS, ITO/n-GraHIL and MXene/n-GraHIL.

FIG. 5 provides a) Luminance versus voltage characteristics of LEDs that have ITO or MXene electrode (inset: optical image of MXene-based green organic LED (left), and schematic of green phosphorescent organic LED device structure (right)). b) Current efficiency versus luminance characteristics (inset: power efficiency versus luminance characteristics), and c) external quantum efficiency versus luminance characteristics of LEDs using ITO and MXene as electrodes. Calculated maximum external quantum efficiency (ZEQE) of organic LEDs with d) MXene, and e) ITO electrode as a function of functional layer thicknesses, f) Optical image of flexible organic LEDs based on MXene electrodes.

FIG. 6 provides Transmittance of a) Ti₃C₂ MXene depending on the spin-coating conditions and b) comparison with ITO.

FIG. 7 provides Air-photoemission spectra of ITO, pristine MXene and vacuum-annealed (200° C.) MXene.

FIG. 8 provides XPS Ti 2p spectra and deconvolutions of a) MXene, b) MXene treated with DI water, and c) MXene treated with 0.01 M HCl.

FIG. 9 provides Changes of sheet resistance R_{S} of MXene/PEDOT:PSS, MXene/GraHIL and MXene/n-GraHIL films as a function of exposure time to humid (RH 60%) air.

FIG. 10 provides Atomic concentration ratio of O and Ti in pristine MXene, MXene/PEDOT:PSS and MXene/n-GraHIL films.

FIG. 11 provides Current density versus voltage of ITO and MXene-based phosphorescent organic LEDs.

FIG. 12 provides the atomic structure of 2D transition metal carbides (MXenes) displayed from the side view. Schematic of carbide MXenes with 3, 5, or 7 atomic layers are represented as M₂C, M₃C₂, and M₄C₃, respectively, where M is an early transition metal (Ti, Mo, V, Nb, or Ta). The material is either comprised of one transition metal in the M-site (top) or ordered double transition metals where one metal occupies the outer M-layers and another metal the central M-layer(s) (bottom). In this study, the outer atom is Mo and the central atom(s) is(are) Ti. All atomic structures are shown with surface terminations (Tₓ=O).

FIG. 13 provides the observed color of exemplary MXene colloids and films. Digital photographs of MXene colloidal dispersions (20 mL) and corresponding free-standing films fabricated by vacuum filtration for a) Ti₂C, b) Ti₃C₂, c) Nb₂C, d) Nb₄C₃, e) V₂C, f) Mo₂C, g) Mo₂TiC₂, h) Mo₂Ti₂C₃ and i) Ta₄C₃. Qualitative description of colors in solution/films, respectively, as follows. Ti₂C: dark purple/green; Ti₃C₂: forest green/dark purple; Nb₂C: blue/golden yellow; Nb₄C₃: grey-brown/grey black; V₂C: green-blue/bronze; Mo₂C: brown/silver; Mo₂TiC₂: orange-brown/light blue-silver; Mo₂Ti₂C₃: grey/dark green-grey; Ta₄C₃: brown/silver-gray. All MXene solutions are dispersed in deionized water with concentrations ranging between 0.01-0.05 mg mL⁻¹. The MXene film diameters are 4 cm.

FIG. 14 provides the ultraviolet-visible-near-infrared (UV-vis-NIR) optical extinction properties of 2D transition metal carbides. Wavelength-dependent extinction coefficient for each MXene calculated by determining the extinction coefficient at each wavelength from 200 to 1000 nm (hv=6.2-1.2 eV).

FIG. 15 provides solid state transmittance and reflectance of MXene thin films. a) Color palette of the (at least partially) transparent Mo₂C, Ti₂C, Mo₂TiC₂, Mo₂Ti₂C₃, Ti₃C₂, V₂C, and Nb₂C thin films with varying degree of optical density fabricated by spray coating of the as-produced MXene colloidal solutions. b) Transmittance (%) at 550 nm for select films (left to right, film #1-5) showing decrease in transmittance with the continuation of spray coating. c) UV-vis-NIR transmittance spectra from 300 to 2500 nm for MXene thin films from panel (a); all samples taken from the 3^{rd} column. d) Schematic illustration of s- and p-polarization in relation to the MXene film on glass along with the axes for “in-plane” and “out-of-plane” polarization of the plasma in red. e-f) Reflectance spectra of the least transparent (5^{th} column) Ti₃C₂ (green), Ti₂C (purple) and V₂C (blue) films spray-cast on a glass substrate. Reflectance spectra were obtained from incident light at 750 from perpendicular to the sample using e) p-polarized and f) s-polarized light.

FIG. 16 provides illustrative peak tuning. Normalized extinction spectra from 350 to 1000 nm for Ti₂C and Ti₃C₂ colloidal solutions in deionized water synthesized via hydrofluoric acid selective etching and tetramethylammonium hydroxide intercalation (HF/TMAOH) or mixed acid selective etching with lithium chloride intercalation media (HF/HCl/LiCl).

FIG. 17 provides Methods used in this study to topochemically synthesize MXenes from layered precursors using hydrofluoric acid (HF) as the etchant and tetramethylammonium hydroxide (TMAOH) as the intercalant or the mixed acid approach utilizing HF/HCl and LiCl as the etchant and intercalant, respectively. a) M₃AX₂ and resulting b) M₃X₂ structures are represented here and the exfoliated material surfaces are populated with c) surface terminations shown as —OH.

FIG. 18 provides a) Zeta potential at neutral pH where the dotted line at −30 mV represents the colloidal stability region (<−30 mV) and b) dynamic light scattering (DLS) intensity distributions for the MXene colloidal solutions.

FIG. 19 provides X-ray diffraction patterns of Mₙ₊₁Aₓ₊₁Xₙ₊ₓ precursors and MXene free-standing films for a) Ti₂C/Ti₂AlC, b) Ti₃C₂/Ti₃AlC₂, c) V₂C/V₂AlC, d) Nb₂C/Nb₂AlC, e) Mo₂C/Mo₂Ga₂C, f) Mo₂TiC₂/Mo₂TiAlC₂, g) Mo₂Ti₂C₃/Mo₂Ti₂AlC₃, h) Nb₄C₃/Nb₄AlC₃, and i) Ta₄C₃/Ta₄AlC₃.

FIG. 20 provides UV-visible-NIR extinction spectra (normalized to mass of material in solution, mL mg⁻¹ m⁻¹) from 200-1000 nm (hv=6.2-1.2 eV) for varying n (top) from a) n=1, b) n=2, c) n=3 and varying M (bottom) where d) M=Ti, e) M=Nb, and e) M=Mo, Ti.

FIG. 21 provides extinction per path length (Ext/l) plotted versus wavelength for serial dilutions of a) Ti₂C, b) Ti₃C₂, c) Nb₂C, d) Nb₄C₃, e) V₂C, g) Mo₂TiC₂, h) Mo₂Ti₂C₃, and i) Ta₄C₃ colloidal solutions. Solution concentrations are less than 0.1 mg/mL.

FIG. 22 provides extinction per path length (Ext/l, m⁻¹) at the λₘₐₓ position versus concentration (mg/mL). The slope of the linear fit is used to calculate the extinction coefficient. V₂C, Mo₂Ti₂C₃, Nb₄C₃, and Ta₄C₃ do not have an extinction peak in the region investigated, therefore Ext/l is taken from 1000 nm.

FIG. 23 provides optical profilometer profile images of a) Ti₂C, b) Ti₃C₂, and c) V₂C thin films deposited on glass substrates.

### DETAILED DESCRIPTION OF ILLUSTRATIVE EMBODIMENTS

MXenes are two-dimensional (2D) transition-metal carbides, nitrides, or carbonitrides that have the formula Mₙ₊₁Xₙ, where M is an early transition metal (e.g., Ti, V, Nb, Mo), and X is C, N, or both. They have metallic conductivity (5000≤σ≤10,000 S cm⁻¹), which is a result of metal-like high free-electron density and a sheet-like structure of individual nanosheets. MXenes have surface hydrophilicity which provides an excellent platform for solution-processing approaches. MXenes can be fabricated using a wet chemical synthesis procedure, which leaves surfaces that are terminated by functional groups such as —OH, —O, —Cl, and —F, which make the MXenes dispersable in polar solvents. Grafting approaches can be used to enable dispersion in non-polar solvents. Due to the 2D structure and high electrical conductivity along with the simple fabrication, MXenes have been explored as ion batteries, sensors, gas storage media and catalysts. Metallic conductivity and hydrophilic surface make MXenes useful as solution-processed transparent conducting electrodes (TCEs) for flexible optoelectronic devices. However, MXene have not been used for supporting an light emitting diode (LED), because the MXene films can be damaged when they are coated with an acidic water-based hole injection layer (HIL). The surface functional groups substantially affect the electrical and electronic properties of MXenes. Oxidation of MXene film significantly degrades its a and decreases its work function (WF). In a TCE, these changes can alter the charge balance in LEDs and significantly decrease their luminous efficiencies, and this result impedes use of Mxene TCEs in optoelectronic devices.

MXene compositions can be, e.g., any of the compositions described in at least one of U.S. patent application Ser. No. 14/094,966 (filed Dec. 3, 2013), 62/055,155 (filed Sep. 25, 2014), 62/214,380 (filed Sep. 4, 2015), 62/149,890 (filed Apr. 20, 2015), 62/127,907 (filed Mar. 4, 2015) or International Applications PCT/US2012/043273 (filed Jun. 20, 2012), PCT/US2013/072733 (filed Dec. 3, 2013), PCT/US2015/051588 (filed Sep. 23, 2015), PCT/US2016/020216 (filed Mar. 1, 2016), PCT/US2020/054912 (filed Oct. 9, 2020), or PCT/US2016/028,354 (filed Apr. 20, 2016), preferably where the MXene composition comprises titanium and carbon (e.g., Ti₃C₂, Ti₂C, Mo₂TiC₂, and the like). MXenes that include transition metals (e.g., Ti, Mo, Nb, Va, Cr) are considered suitable. Each of these compositions is considered an independent embodiment.

Similarly, MXene carbides, nitrides, and carbonitrides are also considered independent embodiments. Various MXene compositions are described elsewhere herein, and these and other compositions, including coatings, stacks, laminates, molded forms, and other structures, described in the above-mentioned references are all considered within the scope of the present disclosure.

In this work, we exploited single-layered Ti₃C₂ MXene as a solution-processed flexible TCE, and used a one-step spin-coating and low-temperature vacuum annealing protocol to obtain MXene electrode films that have high conductivity (up to 11,668 S cm⁻¹ and 98 Ωsq⁻¹), high WF (˜5.1 eV), and good optical transmittance T (up to 85%). To overcome the degradation of the MXene film during device fabrication, we used low-temperature vacuum annealing to modulate the surface of the MXene film, thus protecting the high conductivity and high WF. We also used a chemically neutralized alcohol-based hole-injection layer (HIL) to protect the material from interface oxidation. The effectiveness of these strategies to engineer the surface and interface was demonstrated by analysis of the chemical compositions of MXene films, and by the nearly-ideal charge-injection (hole injection efficiency ˜1) from the modified MXene film to the overlying hole transporting layer (HTL). Phosphorescent organic LEDs with a modified MXene anode showed high efficiency (current efficiency ˜101.9 cd A⁻¹; external quantum efficiency ˜28.5% ph/el %), and show the potential of MXenes as solution-processed flexible TCE for LEDs.

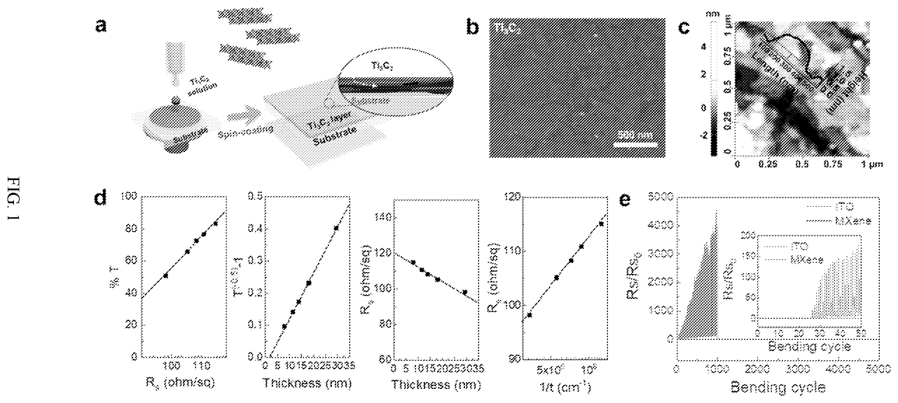

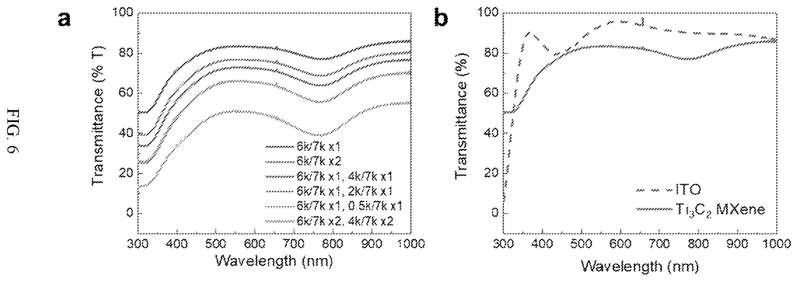

A Ti₃C₂ MXene film was prepared by spin-casting of the Ti₃C₂ solution for flexible transparent electrode application (FIG. 1a). The individual MXene nanosheets in the film overlapped (FIG. 1b); each MXene nanosheet was ˜1 nm thick (FIG. 1c), which closely matches the theoretical value of monolayer Ti₃C₂. T of the MXene film could be widely controlled from 50 to 85% at 550 nm (FIG. 6), and the sheet resistance (R_{S}) of the film could be tuned from 98 to 115 Ω/sq (FIG. 1d) by adjusting its thickness (t) from 29 nm to 7.9 nm. The maximum conductivity=1/(R_{S}·t) of the MXene film was 10,957 S cm⁻¹, which is notably high among flexible conducting films.

In bending tests (bending radius 3 mm), the MXene electrode fabricated on 130-μm-thick PET substrate showed good flexibility, whereas the conventional indium tin oxide (ITO) electrode on PTO substrate cracked upon a few cycles of bending. As a result of the mechanical properties of ITO, its electrical properties also decreased as the number of bending cycles increased. In R_{S} measurements, the MXene electrode on PET did not show any change after 5,000 bending cycles, whereas the ITO on PET showed a sharp decrease increase in R_{S} after 1,000 bending cycles (FIG. 1e).

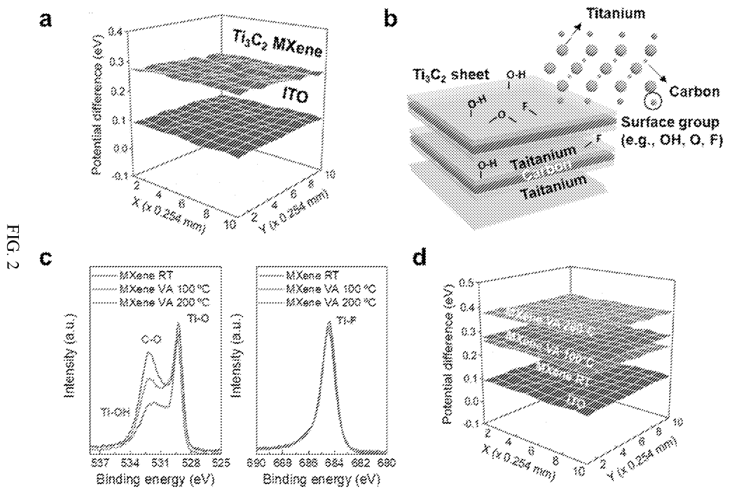

The surface WFs of MXene and ITO electrode films were compared by measuring their spatial surface potentials with Kelvin probe. As-prepared MXene film had a higher WF (˜5.0 eV) than the ITO electrode (˜4.8 eV) (FIG. 2a). The MXene nanosheets are terminated with functional groups such as —OH, —O, —Cl, and —F after the solution preparation process (FIG. 2b). Functional groups on MXene flakes can change the resulting WF by changing the surface dipoles of MXene (i.e., by shifting the Fermi energy level of the metallic semiconductor). Density functional theory (DFT) predicted that the WF of Ti₃C₂ can be in a wide range from 1.6 eV with hydroxyl (—OH) surface termination to 6.25 eV with oxygen (—O) surface termination).

The anode WF strongly influences the charge carriers injection as a result of formation of an energy barrier with the highest occupied molecular orbital (HOMO) energy levels of the overlying organic layer (HIL), so a high WF is desirable for efficient charge injection, which can translate directly to high efficiency of LEDs. The MXene WF of 5.0 eV is higher than those of other flexible electrodes (e.g., graphene: ˜4.4 eV, high-conductivity PEDOT:PSS: ˜4.8 eV), but the usual organic/polymeric HIL has HOMO energy >5.2 eV, so a small energy barrier still exists.

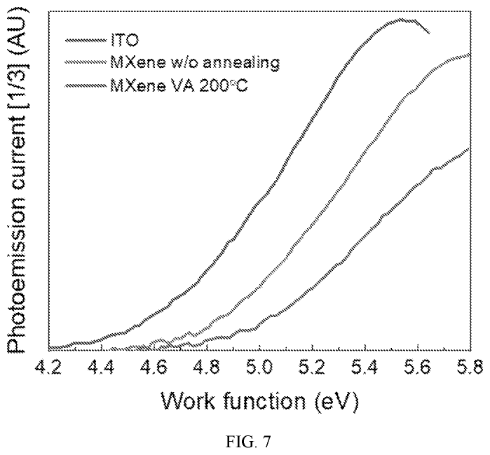

To further increase the WF of MXene, we used vacuum annealing to remove —OH surface functional groups and thereby increase the ratio of —O to —OH surface terminations on the MXene film. To verify the ratio of the functional groups, X-ray photoelectron spectroscopy (XPS) of MXene films were analyzed according to annealing processes (dried at room temperature, vacuum-annealed at 100° C. for 1 h or 200° C. for 2 h) (FIG. 2c). This process noticeably decreased the —OH terminated Ti (534 eV in 0 is spectra) and the C—O contamination (532.2 eV), so the ratio of —O terminated Ti (529.3 eV) and —F terminated Ti (684 eV in F is spectra) on the film surface increased slightly as annealing temperatures and times were increased; as a result the WF of the MXene film increased to ˜5.1 eV (FIG. 2d). The air-photoemission measurement provided further evidence that the WF increased in the MXene film after vacuum annealing (FIG. 7). Also, the low-temperature vacuum annealing and concomitant altered surface chemical composition increased the conductivity of the MXene film to 11,668 S cm⁻¹ (R_{S}=108 Ω/sq) after annealing in vacuum at 200° C. for 2 h.

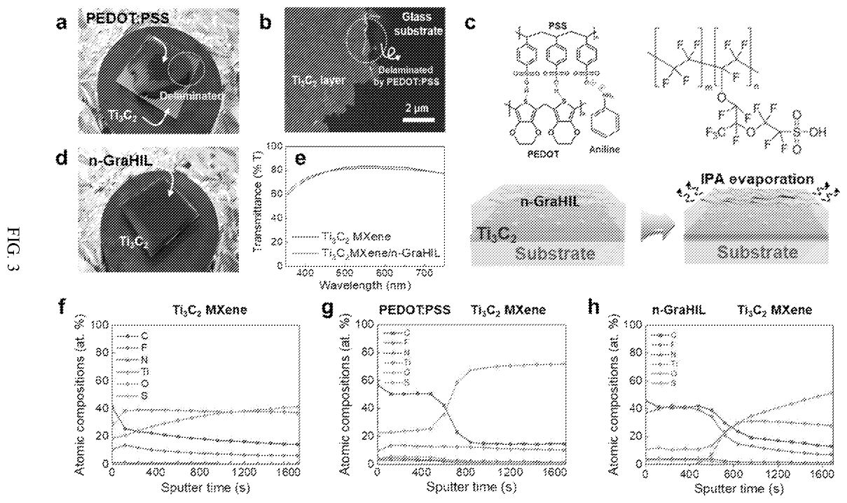

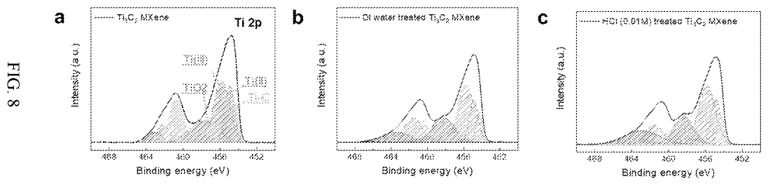

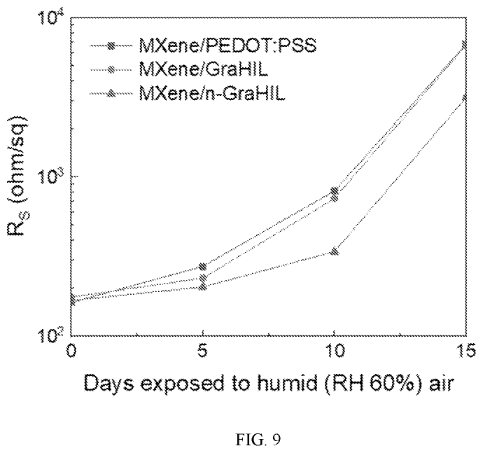

A MXene electrode should not react with the overlying solution-processed HIL. However, humid and acidic environments oxidize Ti₃C₂ MXene. The influence of water and acid permeation on the chemical compositions of Ti₃C₂ MXene film was investigated by XPS analysis. Composition changes of Ti₃C₂ were observed after water and acid exposure on the MXene film (FIG. 8). The TiO₂ peak centered at 458.5 eV in the XPS Ti 2p spectrum strengthened after exposure to deionized water or hydrochloric acid (HCl); these changes imply that Ti₃C₂ on the surface of the film is oxidized. This result also indicates that the acidity of HIL also accelerates the oxidization of Ti₃C₂ MXene electrode films, so the electrical conductivity of the electrode decreases (FIGS. 8, 9). Also, the application of the conventional polymeric HIL, PEDOT:PSS to MXene-coated substrate caused Ti₃C₂ sheets to delaminate from the substrate (FIG. 3a). This result could be also attributed to permeation of water into the Ti₃C₂ layers due to hydrophilic —OH and —O surface terminations of the Ti₃C₂ sheets, and to the acidic nature of PEDOT:PSS that could oxidize the MXene film. XPS depth profiles of bare Ti₃C₂ and PEDOT:PSS-coated Ti₃C₂ revealed that water-dispersed PEDOT:PSS diffused throughout the Ti₃C₂ film (FIG. 3f, g). Judging from the atomic concentrations of C and S, the depth profile was divided into HIL and Ti₃C₂ MXene regions (FIG. 3f-h). The atomic concentration of oxygen in the MXene film (where the concentration of C is reduced) increased significantly compared to that of bare Ti₃C₂ after spin-coating of PEDOT:PSS on MXene film (FIG. 3g).

To eliminate the water/acid permeation and delamination of the MXene electrodes, a chemically neutralized polymeric HIL (n-GraHIL) was introduced in LED applications. The conventional PEDOT:PSS was neutralized by incorporating Lewis-basic aniline molecules into the PEDOT:PSS solution to chemically coordinate acidic polystyrene sulfonic acid (PSS) moieties in the PEDOT:PSS (FIG. 3c), so the pH of this n-GraHIL was tuned to be near 7. The n-GraHIL also contains a perfluorinated polymer that has low surface energy and high ionization potential, thereby additionally developing a gradient WF inside the HIL (surface WF: ˜5.95 eV) and yielding efficient hole injection into the emitting layer (EML). The water content in the n-GraHIL solution was reduced by replacing water with alcohol as a solvent for the polymer blends so that water permeation into the electrode film could be reduced during the solution process compared to the process that used water-based solution.

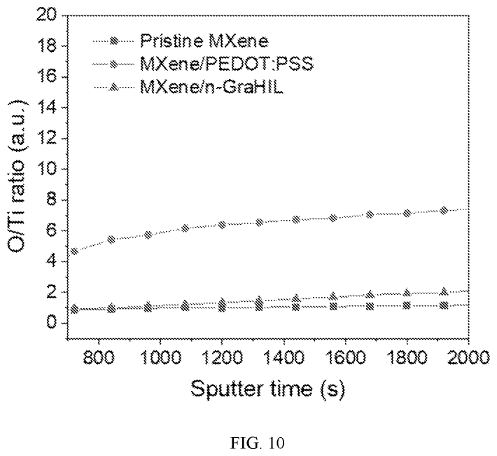

The pH-neutral and alcohol-based HIL was used to avoid oxidation, and it did not cause delamination of Ti₃C₂ layers during spin-coating (FIG. 3d). The n-GraHIL film formed uniformly on the Ti₃C₂ electrode film and did not show significant effect on its T (FIG. 3e). These results can be attributed to the neutral environment and to the faster evaporation of alcohol-based solvent than water during HIL formation on the MXene film (FIG. 3c). The fast solvent evaporation of n-GraHIL did not allow time for diffusion into Ti₃C₂ sheets, or for surface oxidation of electrode film. The atomic concentration of oxygen and the ratio of O/Ti did not change much from the XPS depth profile of bare Ti₃C₂ (FIG. 3h, FIG. 10).

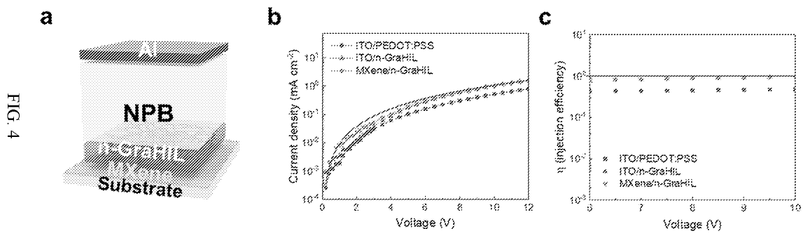

To evaluate the hole-injection capability from the MXene anode/n-GraHIL stack, we fabricated hole-only devices (HODs) (FIG. 4a). At low voltage, the voltage-current density curves of the HODs with Ti₃C₂ MXene anode showed higher hole-current density at a given applied bias than did the HODs that used a conventional ITO anode; this result can be attributed to MXene anode's higher WF (˜5.1 eV) than that of ITO (˜4.8 eV) (FIG. 4b). The hole-injection efficiency of MXene/n-GraHIL was calculated by comparison with a theoretical space charge-limited current (SCLC) model and Poole-Frenkel equations (FIG. 4c). The MXene/n-GraHIL showed hole-injection efficiency ˜1, which means that Ohmic-like hole injection is achievable using an MXene anode with engineered surface and interface chemical composition.

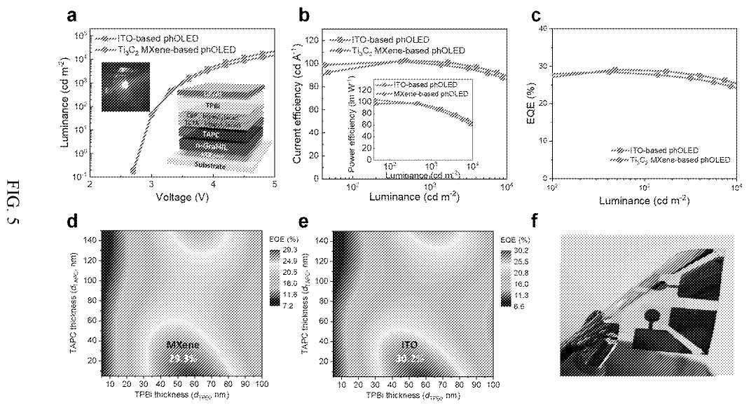

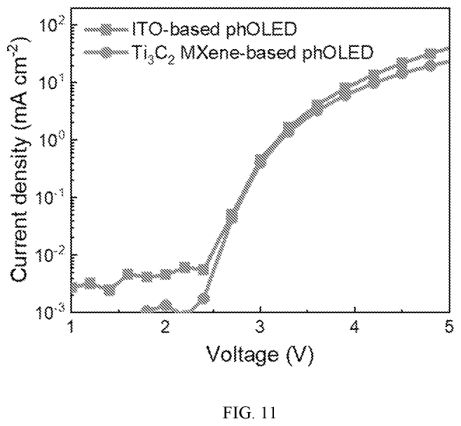

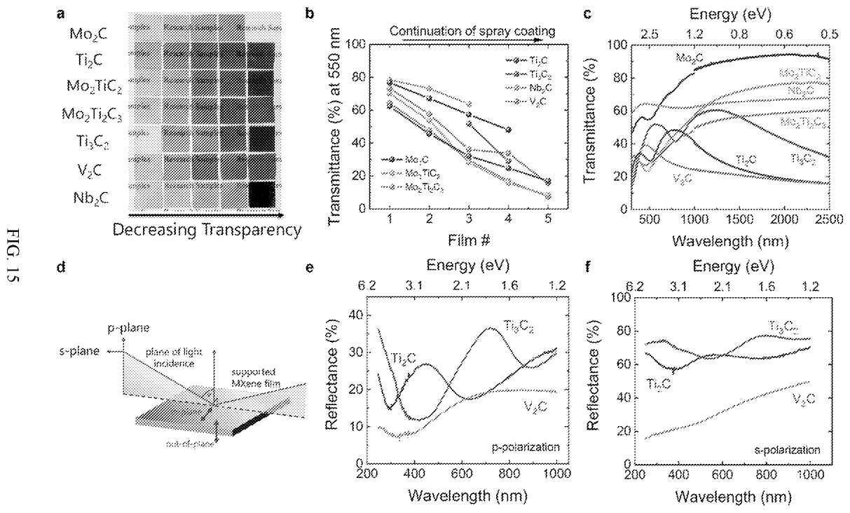

We also fabricated green phosphorescent organic LEDs that had solution-processed Ti₃C₂ MXene film as a TCE (FIG. 5a). They had high current density and luminance characteristics at a given applied bias (FIG. 5a, FIG. 11). The maximum current efficiency was 102.0 cd A⁻¹, maximum power efficiency was 103.7 lm W⁻¹, and maximum external quantum efficiency was 28.5%; these are comparable to or slightly higher than those of conventional rigid ITO-based devices (103.0 cd A⁻¹, 96.9 lm W⁻¹ and 29.1%) (FIG. 5b,c). These results are very close to the maximum EQE values calculated using optical simulation (29.3% for MXene-based devices; 30.2% for ITO-based devices) (FIG. 15d, 15e). Therefore, MXene provides efficient hole-carrier injection into the emitting layer of LEDs, and thereby yields a favorable internal charge balance in the LEDs. Also, bright, green-emitting, flexible LEDs that used a flexible PET/MXene anode were demonstrated (FIG. 5f); this success highlights the great feasibility of using an MXene anode in flexible optoelectronics.

Thus, we demonstrated the feasibility of single-layered Ti₃C₂ MXene as a solution-processed flexible TCE for LEDs and achieved high-efficiency organic LEDs based on a MXene anode for the first time by using precise surface and interface engineering. MXene electrode produced using a simple spin-coating and low-temperature post annealing process had highly-desirable electrode properties of high WF (˜5.1 eV) and high electronic conductivity (up to 11,668 S cm⁻¹), as well as good T (up to 85%). Organic LEDs with the surface-modulated MXene anode and neutralized polymeric HIL achieved high current efficiency (˜102.0 cd A⁻¹), power efficiency (103.7 lm W⁻¹) and EQE (28.5% ph/el), which approach the theoretical maxima in this device structure. The outstanding results of MXene film and the MXene anode-based flexible organic LEDs demonstrate the strong potential of the solution-processed MXene TCE for use in next-generation optoelectronics that are produced using a low-cost solution-processing technology.

Experimental Section

Ti₃C₂synthesis: First, 2 g of Ti₃AlC₂ MAX phase (<38 μm) was slowly added over the course of 10 min to 40 mL of etchant solution (24 mL hydrochloric acid (HCl, 37 wt. % Fisher Scientific), 12 mL deionized H₂O, 4 mL hydrofluoric acid (HF, 48-51 wt. % Sigma Aldrich)). The reaction was stirred at 35° C. for 24 h using a Teflon magnetic stir bar. After the selective etching reaction, the sediment was washed by repeated centrifugation (5 min, 3500 rpm, 150 mL deionized H₂O), the acidic supernatant was decanted, and the process was repeated until the pH reached neutral (˜6). Then 2 g of lithium chloride (LiCl, Sigma Aldrich) was dissolved in 100 mL of deionized H₂O and added to the multilayer MXene sediment. The solution was stirred for 12 h at ambient temperature. The solution was washed with repeated centrifugation (5 min, 3500 rpm, 150 mL deionized H₂O) and the supernatant was decanted until a dark supernatant was observed. Then the solution was centrifuged for 1 h at 3500 rpm and the dilute green supernatant was decanted. The swollen sediment was re-dispersed with 150 mL of deionized H₂O and centrifuged for 10 min at 3500 rpm to isolate the MXene supernatant from the sediment. To optimize the flake size, the MXene supernatant was centrifuged for 30 min at 3500 rpm. The final supernatant was used in fabrication of TCEs.

Ti₃C₂ film preparation: Glass substrates were immersed sequentially in acetone and isopropyl alcohol (IPA) baths and sonicated for 10 min, each. The substrates were surface-treated by ultraviolet light and ozone for 10 min. Then 250 μL of the Ti₃C₂ solution (14 mg/mL) was deposited on the substrate and allowed to equilibrate for 30 s, then it was dispersed by spin-coating at 6000 rpm for 30 s, then at 7000 rpm for 5 s to yield in a thin, conductive electrode film. All films were dried at room temperature or vacuum-annealed 100° C. for 1 h or 200° C. for 2 h. The films were stored in a nitrogen glovebox at room temperature.

Characterizations: The surface topographic images of MXene films were obtained by atomic force microscopy (NanoScope, Digital Instruments) and field-emission scanning electron microscopy (MERLIN compact, ZEISS) at the Research Institute of Advanced Materials, Seoul National University. The optical transmittances of MXene films were measured using an ultra-violet (UV) absorption spectroscopy (Lambda 465, PerkinElmer, Inc.) and the sheet resistances of the films were obtained by 4-point probe measurement combined with a Keithley 2400 source meter. The thickness was calculated using Beer-Lambert law calibration curve (Ti₃C₂ absorbs 3% of visible light (at 550 nm) per nanometer thickness; i.e., has an absorption coefficient of 1.1×10⁻⁵ cm⁻¹. Surface potentials were obtained using a Kelvin probe and air photoemission system (APS) (KP Technology Ltd.). For surface composition analysis, X-ray spectroscopy spectra were analyzed using a Micro X-ray/UV photoelectron spectroscopy system at the Korea Basic Science Institute.

Device fabrications: Conducting polymer hole-injection layers (neutralized-gradient WF hole injection layer (n-GraHIL) or poly(3,4-ethylenedioxythiophene) polystyrene sulfonate (PEDOT:PSS)) were spin-coated on patterned Ti₃C₂ substrates to give 100-nm-thick film. To fabricate organic LEDs, the substrate/anode/HIL samples were transferred to a thermal evaporator for deposition of organic layers and metal electrodes. The organic layers of TAPC (15 nm)/TCTA: Ir(ppy)₂acac (5 nm, 97:3 by volume)/CBP: Ir(ppy)₂acac (5 nm, 96:4, by volume)/TPBi (55 nm), (TAPC=4,4′-cyclohexylidenebis(N,N-bis(4-methylphenyl) benzenamine), TCTA=Tris (4-carbazoyl-9-ylphenyl) amine, CBP=4, 4′-Bis(N-carbazolyl)-1, 1′-biphenyl, TPBi=2, 2, 2-(1, 3, 5-benzenetriyl)tris-(1-phenyl-1H-benzimidazole)), were deposited using a thermal evaporator under 5.0×10⁻⁷ Torr. Metal electrodes of lithium fluoride, LiF (1 nm)/aluminum (100 nm) were deposited sequentially.

Additional Disclosure—Optical Properties of MXenes

Also disclosed are compositions, systems, and methods related to the optical properties of MXenes. The observed optical phenomena (without being bound to any particular theory or embodiment) span the ultraviolet to infrared and include intraband transitions and plasma excitations. The spectral features, involving excitation of the plasma, can provide an optical readout of the composition-dependent carrier concentration, revealing even subtle changes due to surface chemical modification. Also without being bound to any particular theory or embodiment, the high carrier concentration found in MXenes differentiates them from other known 2D materials, and (also without being bound to any particular theory or embodiment) MXenes host optically active plasmon resonances that naturally span the UV to near-IR, as a function of composition. This discovery thus benefits the interpretation of 2D materials spectroscopy and further confirms the utility of MXenes as optoelectronic building blocks.

MXene Synthesis and Processing

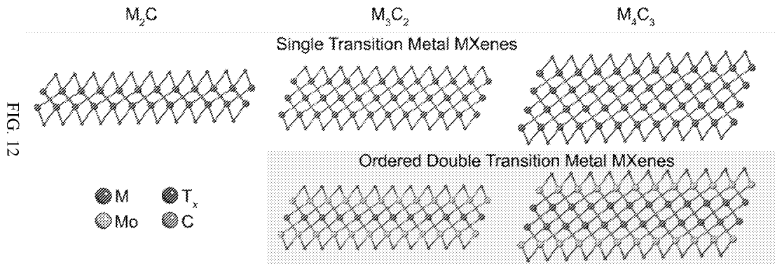

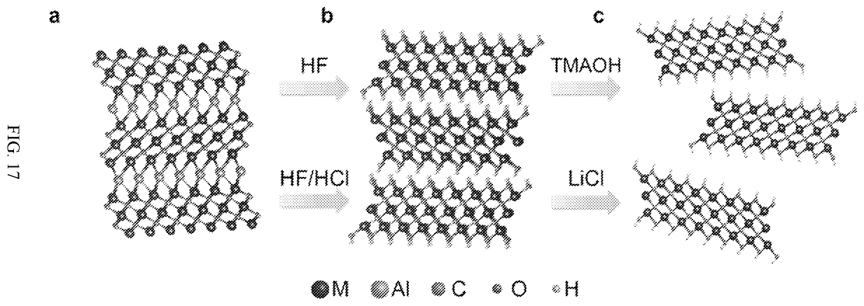

MXenes were produced with varying M (Ti, V, Nb, Mo, Ta) and n (M₂C, M₃C₂, and M₄C₃), shown schematically in FIG. 12, by selective etching of aluminum or gallium from layered precursors using hydrofluoric acid (HF) following previously reported synthesis routes (details provided elsewhere herein). Using tetramethylammonium hydroxide (TMAOH) as the intercalant, multilayered MXenes were delaminated into single to few-layered flakes in deionized water (FIG. 17).

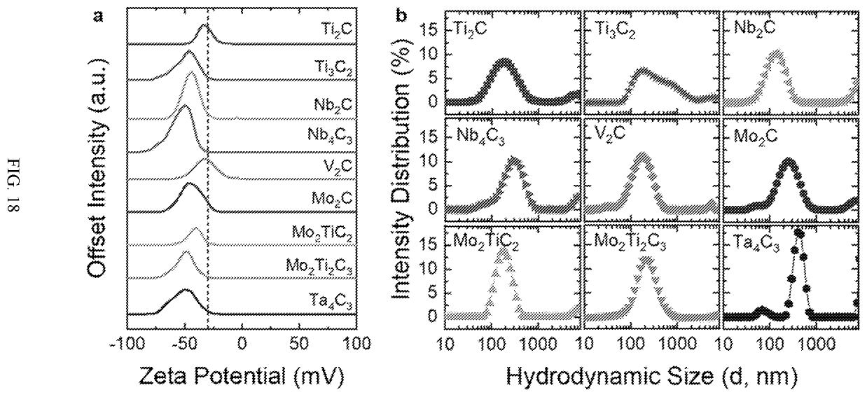

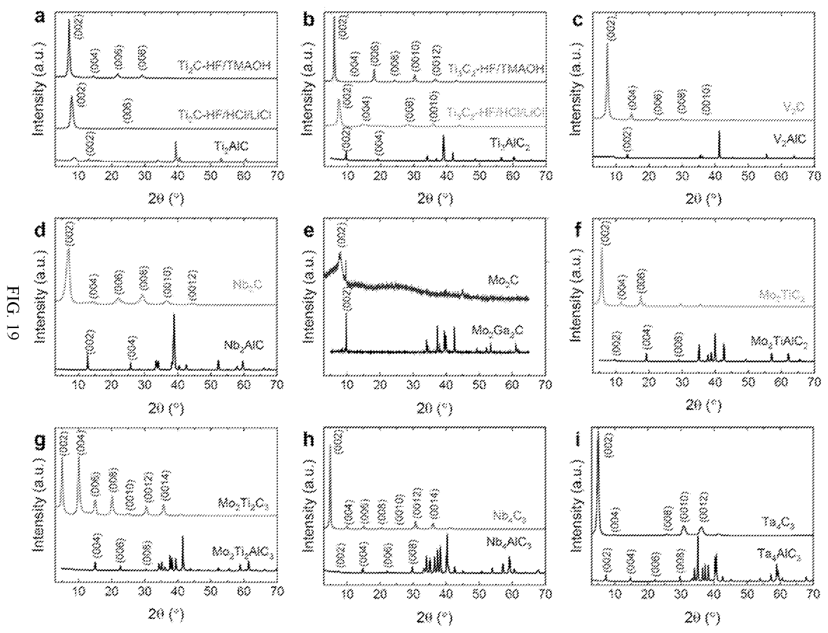

Characterization of the colloid revealed a negative zeta potential below −30 mV at neutral pH and particles with lateral size averages ranging from 100 to 500 nm (FIG. 18). X-ray diffraction (XRD) patterns of the free-standing films, produced by vacuum filtration of the MXene colloidal solution, show that there is a shift of the (001) peak to lower 20 after etching and intercalation, and that there are no residual peaks of corresponding precursor. This is indicative of complete A-element removal and conversion to MXene, in addition to significant preferential ordering of the 2D flake basal plane within the film (FIG. 19).

Observed Colors of MXenes

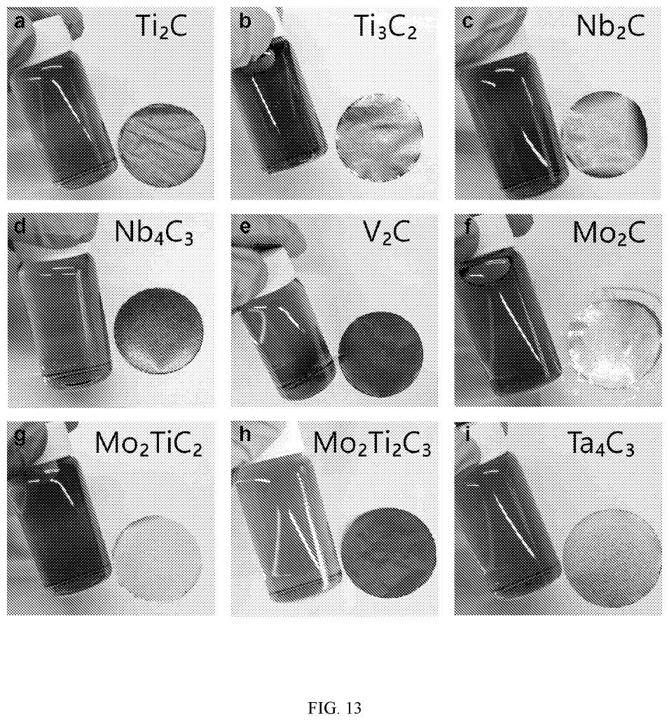

Depending on the MXene composition, the colloidal solutions vary in perceived color and the corresponding free-standing films exhibit complementary colors (FIG. 13). For all MXenes included in this study, the X-element is carbon, however, even the compositions with the same M-site transition metal and different n exhibit varying colors, displaying both composition and structural dependence of the observed optical properties. For example, Ti₂C resembles a dark purple color in solution, and a green film color (FIG. 13a), while the Ti₃C₂ solution is forest green and the film is dark purple (FIG. 13b).

Quantifying and Interpreting Transmission and Reflectance Spectra

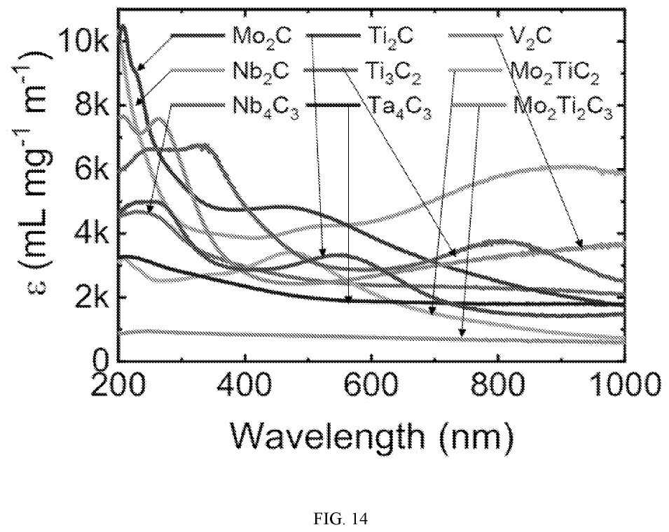

Ultraviolet-visible-near-infrared (UV-vis-NIR) transmission and reflectance spectroscopies allow quantification of the differences between MXenes observed by eye and extend the wavelength range beyond human eyesight. Extinction spectra, which are the sum of the absorbance and scattering (reflection losses), were measured for solutions and films, and the reflectance was measured for films (FIGS. 14 and 15).

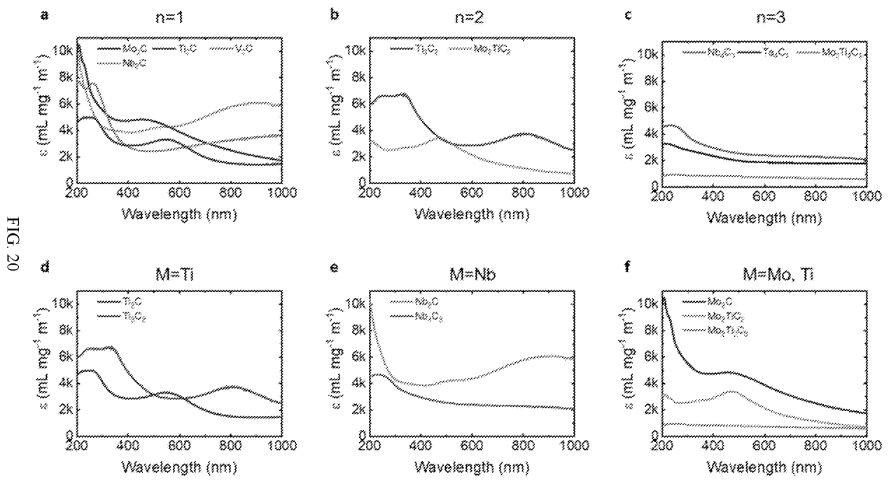

In order to compare the intensity of the diverse optical phenomena hosted in different MXene compositions on an absolute, quantitative scale, the gravimetric extinction coefficients, ε [mL mg⁻¹ m⁻¹], were measured over a range of wavelengths spanning the UV-vis-NIR spectrum (FIG. 14a). To facilitate systematic comparisons within a family of structures, the reported solution spectra are plotted separately as a function of varying n (1, 2, 3) and varying M (Ti, Nb, or Mo, Ti) in FIG. 20. For nearly all MXenes in the set, the intensity of the extinction features in the UV region varied over a relatively narrow range (˜3000-10000 mL mg⁻¹ m⁻¹), one which encompasses values previously observed for TiO₂ nanoparticles, consistent with the high density of intra-band transitions in this energy regime for both material classes. In the visible-to-near-IR region, composition and structure variation resulted in strong extinction peaks that with tremendous spectral diversity: taken as a set, these peaks span the entire spectral range, with extinction coefficients only slightly lower than those in the UV. Ti₃C₂ exhibited an extinction peak in the NIR region (λₘₐₓ=800 nm), as reported previously in the literature. Reducing n by one to form Ti₂C, resulted in qualitatively similar spectrum, however with the prominent extinction peak shifted to higher energy, with λₘₐₓ of 542 nm. Comparing either M₂C or M₃C₂ structures, the complete or partial replacement of Ti with Mo similarly resulted in shifts to higher energy, with λₘₐₓ instead observed at 450 nm (Mo₂C) or 476 nm (Mo₂TiC₂). Nb₂C showed a broader, more intense, extinction feature, peaking in the NIR region (λₘₐₓ of 915 nm), whereas V₂C exhibits no peaks in the visible. Nb₄C₃ and Ta₄C₃ did not have a distinct extinction peak in the spectral range explored.

Solid State MXene Optical Transmission and Reflectance

Further qualitative differences between MXene spectra are observed by measuring both the transmittance and reflectance of solid thin films and extending the spectral range further to the NIR, thus providing insight into the origins of the spectral variation. Thin films were prepared by spray coating the Mo₂C, Ti₂C, Mo₂TiC₂, Mo₂Ti₂C₃, Ti₃C₂, V₂C, and Nb₂C colloids onto glass substrates (FIG. 15a). The color of the thin films observed at normal incidence over a white background matches the solution color until visible transparency is lost, at which point the films have a brown or black hue. By increasing the spray coating amount, thus increasing the film thickness, the visible transparency (550 nm) is decreased (FIG. 15b). In the transmission spectra of these films, shown in FIG. 15c, a distinction emerges between MXenes that exhibit an increase in transmittance with wavelength (positive slope) over most of the NIR (Mo₂C, Mo₂TiC₂, Mo₂Ti₂C₃ and Nb₂C) versus those that exclusively decrease in transmittance (negative slope) over the same range (V₂C, Ti₂C, and Ti₃C₂). Among those MXenes with an exclusively negative slope, the onset of the decrease in transmittance is different for each material, occurring at ˜600 nm (2.07 eV) for V₂C, ˜850 nm (1.46 eV) for Ti₂C, and ˜1250 nm (0.99 eV) for Ti₃C₂. V₂C and Ti₂C have been shown previously to exhibit high DC conductivity, roughly on the same order of magnitude as Ti₃C₂,^{[31,32]} where electronic transport studies have measured a carrier concentration as high as 3×10²² cm⁻³. Given their similarities in terms of structure and synthesis, one can speculate that their respective carrier mobilities are similar (i.e. within an order-of-magnitude), therefore V₂C, Ti₂C, and Ti₃C₂ may share a similar carrier concertation, thus comprising a set of MXenes for which we anticipate metal-like optical behavior, a priori. In contrast Mo₂C, Mo₂TiC₂, and Mo₂Ti₂C₃ processed in a similar manner possess a much lower carrier concentration, in the range of 2-8×10²⁰ cm⁻³. For Ti₃C₂, the negative transmittance slope has been shown to arise from metal-like reflectivity, occurring in the vicinity of the wavelength where the real dielectric crosses over to negative values; such an optical response is captured by modeling this relatively high concentration free-carrier plasma as a Drude oscillator. Without being bound to any particular theory or embodiment, one can speculate that, for films measured in FIG. 15c, the feature of a sustained, deeply negatively sloping transmittance with increasing wavelength, beginning at near-IR or shorter wavelengths, is generally suggestive of a high concentration plasma capable of strong reflectivity; consequently, we posit that this optical signature can be used as a preliminary means to rapidly sort novel MXenes when the carrier concentration is unknown. Consistent with this classification scheme, Mo₂C, Mo₂TiC₂ and Mo₂Ti₂C₃ do not show a negative slope even out to 2000 nm, as expected given their known lower carrier concentration. Nb₂C is similarly missing this optical hallmark of high conductivity and we note that, although the carrier concentration has yet to be quantified, the DC sheet conductivity is below the detection limit of a hand-held voltmeter, unlike V₂C, Ti₂C, and Ti₃C₂.

To understand additional qualitative similarities in the optical response of the three most conductive MXenes, reflectance spectra, shown in FIG. 15e-15f, were taken. A relatively oblique angle of incidence of 750 from perpendicular was employed to accentuate the difference between s- and p-polarized light (illustrated in FIG. 15d). The spectra of Ti₂C and V₂C agree with what was already known for Ti₃C₂: a strong and increasing reflectance coincides with the onset of the negative slope of transmittance, again consistent with the classification of these three MXene as examples of high carrier concentration. The qualitative similarities among this set also extend to the characteristic UV-vis-NIR features discussed above: a strong reflectance peak is observed at 720 nm for Ti₃C₂, while Ti₂C is seen at 442. For V₂C a similar feature may be partially resolved around ˜250 nm, at the same wavelength of the UV shoulder seen in in the extinction spectra of FIG. 14.

The primary aim of this report is to survey the broad range of optical responses hosted by MXenes, however the juxtaposition of multiple examples presents the opportunity to speculate on the origins of the prominent extinction/reflection peaks, especially those observed in the UV-vis-NIR range for V₂C, Ti₂C and Ti₃C₂. Two competing hypotheses are: 1) plasmonic resonance or; 2) interband transitions. The vast majority of studies on MXene optical properties have only addressed Ti₃C₂, which density functional theory (DFT) suggests that optically active interband transitions could be associated with the ˜800 nm extinction peak. On the other hand, a study employing scanning transmission electron microscopy with electron energy loss spectroscopy (STEM-EELS) found a loss peak for Ti₃C₂ nanoflakes at 1.7 eV (730 nm) that is independent of the flake size, has uniform intensity across the lateral dimensions of the flake, and shifts to higher energy in proportion to increased carrier concentration; on this basis, the authors convincingly assigned the loss peak to plasmon resonance. The nearly perfect correspondence between the energy of the EELS peak in individual nanoflakes and the p-polarized reflectance peak in Ti₃C₂ films at 720 nm (FIG. 15e) is highly suggestive of a common mechanism, i.e. that both transitions may be plasmonic in origin.

In further support of the origin of the peak being plasmonic for the observed reflectance or extinction properties, an ultrafast transmittance and reflectance study was published during the preparation of this manuscript in which it is argued that the observed transient response in the region of the 800 nm peak is consistent with plasmonic behavior and distinct from the kinetics observed in the region of the known interband peaks. Also in favor of the plasmonic hypothesis, we note that the extinction peak exhibits a large, continuously tunable 100 nm blue shift with little change in peak-shape when Ti₃C₂ (or Ti₂C) films are used as cathodes in electrochromic devices, as expected for a plasmon subject to a voltage-driven increase in electron density. If the effect was instead due to state-filling of a typical interband transition, our a priori expectation would be of a significant change in peak shape as the lowest energy states are filled first, depleting the red-edge.

Whether due to free-carrier, plasmonic or interband excitations, the strong qualitative similarities in both transmittance and reflectance between Ti₃C₂, Ti₂C and V₂C (FIGS. 14 and 15) suggest that the same mechanisms are at work for all three, varying only in the specific frequencies observed. Supporting this, and consistent with the plasmonic hypothesis for the UV-vis-NIR range peaks, the energy onset of reflectivity (0.99, 1.46 and 2.07 eV, respectively), putatively due to free-carrier, Drude-type reflectivity follows the same trend as the peak position (1.7, 2.8 and 5 eV, respectively), increasing in the order Ti₃C₂<Ti₂C<V₂C. If all other relevant factors are equal and assuming a simplistic free-carrier, Drude model, both the onset of free-carrier reflectivity and the frequency of plasmonic resonances should monotonically increase with plasma frequency; plasma frequency is in turn proportional to the square root of the free carrier concentration. Given that the comparison between Mₙ₊₁Xₙ structures of different n may be complicated by structural effects on plasmon resonance, we restrict our focus to the only two highly conductive MXenes of the same n in our study: V₂C and Ti₂C. If the UV-vis peaks represent the same plasmonic mode, the square of the ratio of the V₂C to Ti₂C resonance frequencies, approximately 5, would be the predicted ratio of their respective carrier concentrations.

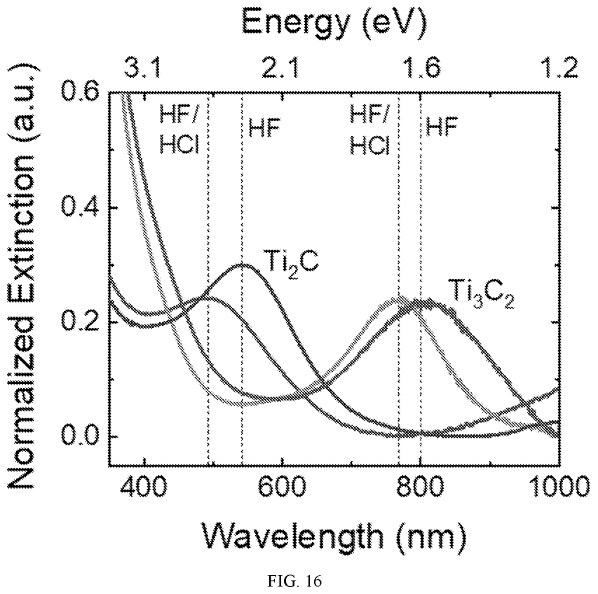

The plasmonic hypothesis leads to another testable prediction: that increases in carrier concentration due to modified surface chemistry should lead to spectroscopically observable peak shifts to higher energy. To test this, we varied the etchant and intercalant chemistries. The materials presented up to this point were synthesized in a HF etchant and delaminated using TMA⁺; however, it is known from previous studies that the etching and processing conditions have direct implications on the quality of flakes produced, surface terminations present, and the resultant carrier transport properties including mobility and carrier concentration. By changing the synthesis and processing approach to a mixed acid etchant (HF/HCl) and LiCl intercalation media, the prominent extinction/reflection peaks of Ti₃C₂ and Ti₂C exhibit blue shifts of, respectively, ˜0.065 eV (from 800 nm to 768 nm for Ti₃C₂) and ˜0.23 eV (from 542 nm to 492 nm for Ti₂C) (FIG. 16) consistent with an increase in carrier concentration, if the peaks are plasmonic in origin. In the case of Ti₂C, the low energy onset of increased extinction, assigned to free-carrier plasma oscillations above, similarly shifts to higher energy (from 850 nm to below 800 nm) as expected for an increase in carrier concentration.

Four-point probe sheet resistance measurements at room temperature and ambient atmosphere quantify the increase in DC conductivity for Ti₂C: films synthesized from HF/HCl/LiCl measure 1320±81 S/cm compared to 142±12 S/cm when synthesized from HF/TMAOH. Similar DC conductivity trends are present for Ti₃C₂ synthesized from two approaches where HF/HCl/LiCl improved the electronic conductivity from 411±49 S/cm (HF/TMAOH) to 7310±120 S/cm. The correlation of increasing DC conductivities and plasmon resonance frequencies are consistent with the plasmonic interpretation of the spectra, however, the DC conductivity increases more than would be expected based on the resonance shifts alone, which could indicate a significant improvement in mobility as well.

Summary

This study demonstrates the diversity of MXene optical properties using representative compositions of this family of 2D materials. Analysis of the NIR reflectivity of the different classes of MXenes suggests a broad classification distinguishing between high-versus low-carrier concentration materials based on the optical spectra, and we can preliminarily identify their plasmonic features. Given the range of MXene compositions (including, e.g., i-MXenes, double-M MXenes, nitrides, carbonitrides and M₅X₄ structures), the MXene family presents attractive optical properties. For example, Nb₂C exhibits a strong extinction coefficient in the NIR, which is also the biological transparency region, implying a potential for applications in photothermal therapy or use in other biomedical applications where strong IR interactions are needed. Nb₄C₃, Mo₂Ti₂C₃, Ta₄C₃ and, V₂C, have relatively low extinction (less than 3000 mL mg⁻¹ m⁻¹) at 550 nm implying low optical losses for photonic applications such as transparent conductors. Such optical properties can be fine-tuned by changing surface chemistry, C/N ratio in carbonitride MXenes, or mixing M-elements in solid solutions. The plasmonic hypothesis of the UV-vis-NIR extinction features also provides potential understanding of the tunability mechanism of optical properties possible through applied voltage (electrochromism) or by changes in processing conditions. Combining the tunable optical properties with other useful features of MXenes, such as electronic conductivity (Ti₃C₂ has a higher conductivity compared to any other solution-processable 2D material reported to date), extreme mechanical strength, light-to-heat conversion, and hydrophilicity, which potentially allows the printing of devices directly from aqueous solutions with no additives, numerous applications can be envisioned. In particular, applications which strive to control or modulate light, such as electrochromic devices, color filters, or metamaterial and cloaking devices, may be interesting to pursue with MXenes, depending on the spectral region of interest.

Experimental Section

Synthesis of MXenes from ternary layered carbides

All materials investigated in this study (Ti₂C, Ti₃C₂, Mo₂C, Mo₂TiC₂, Mo₂Ti₂C₃, V₂C, Nb₂C, Nb₄C₃, and Ta₄C₃) were topochemically synthesized from a ternary layered carbide (MAX phase or similar) containing either Al or Ga₂ as the A-layer (Ti₂AlC, Ti₃AlC₂, Mo₂Ga₂C, Mo₂TiAlC₂, Mo₂Ti₂AlC₃, V₂AlC, Nb₂AlC, and Nb₄AlC₃, respectively) using hydrofluoric acid (HF) as the etchant. The ratio of MAX to etchant was kept consistent (1 g MAX: 10 mL of etchant), however the etching times and temperatures varied for each composition.

Delamination

Following synthesis and washing to neutral pH, powders were dried overnight in a desiccator before performing delamination. Intercalation was performed by mixing 1 g of multilayered powder in 20 mL of deionized water. 1 mL of tetramethylammonium hydroxide (Sigma Aldrich, 25 wt. % in water) was added to the solution. The mixture was stirred at 200 rpm for 12 hours (2 h in the case of Ti₂C). After the reaction, the solution was washed to neutral pH by repeated centrifugation (5000 rpm, 30 minutes) and the sediment was re-dispersed with 50 mL deionized water. The solution was bath sonicated for 1 h and then centrifuged at 3500 rpm for 1 h. The supernatant was kept for analysis. Optical extinction characterization and film fabrication was conducted immediately to ensure minimal material oxidation or degradation.

Effect of Synthesis/Delamination Method

To compare etching methods, Ti₃AlC₂ and Ti₂AlC were etched following mixed acid (HF/HCl) in a 6:3:1 volume ratio (10 mL) (HCl:H₂O:HF) per 0.5 g of MAX. The reaction proceeded for 24 hours at room temperature. Ti₂AlC was added under an ice bath. The powders were washed to neutral pH with repeated centrifugation at 3500 rpm or 5 min cycles and the acidic supernatant was decanted. Intercalation medium was prepared by dissolving 1 g of lithium chloride (LiCl) in 50 mL deionized water and was added to the neutralized MAX/MXene sediment. The sediment was agitated by hand shaking for 15 minutes. Again, the mixture was washed with repeated centrifugation (10000 rpm for 10 minutes, 50 mL deionized water, 5 washing cycles) followed by decanting the clear-dilute green (Ti₃C₂) or purple (Ti₂C) supernatants. Finally, the sediment was re-dispersed with deionized water and centrifuged at 3500 rpm for 1 h. The supernatant after centrifugation was used for optical studies. Four-point probe resistance measurements (ResTest v1, Jandel Engineering Ltd., Bedforshire, UK) probe distance of 1 mm) were conducted on free-standing films at 5 points and the average is reported.

Thin Film Fabrication

Before film deposition, glass slides with 2.5 cm×2.5 cm dimensions were cleaned by bath sonication (40 kHz, Branson Ultrasonic Cleaner, Branson Ultrasonics, USA) in Hellmanex III detergent for 10 minutes. The substrates were cleaned by sequential rinsing in a deionized water bath, followed by an ethanol bath, followed again by a deionized water bath and dried. To make the surface more hydrophilic before depositing MXene, the glass slides were exposed to mixed O₂/Ar (3/5 sccm) plasma treatment at 150 W for 5 minutes. Thin films were prepared by spray coating technique using a Master Airbrush operating 20 cm at ˜45° angle from the substrate and solution concentrations between 1-2 mg mL⁻¹. Spray coating was conducted serially, systematically removing films with every 2-4 mL of solution (depending on starting concentration).

Extinction Coefficient Determination

A known volume of MXene supernatant solution (e.g., 10 mL) was filtered through a Celgard membrane (0.09 μm pore size, 3501 Cated PP, Celgard, USA), vacuum dried at ambient temperature for 12 hours, and the mass of the material was measured. From the measured mass, the concentration in solution was calculated in mg mL⁻¹. Directly following delamination and to prevent oxidation, the as-prepared solutions were serially diluted in the range available for UV-vis-NIR extinction testing (extinction <2). Optical extinction spectra were collected using a 10 mm path length quartz cuvette and a blank composed of deionized water. The extinction coefficient was calculated from the slope of the Ext/l versus concentration of the solution via the Beer Lambert law (Ext=εCl), where Ext is the extinction measured in UV-vis-NIR spectrophotometry, C is the concentration of the solution in mg mL⁻¹, and l is the path length of the cuvette. This allows for the calculation C of the solution if ε and l are known and Ext is measured. V₂C, Mo₂Ti₂C₃, Nb₄C₃, and Ta₄C₃ do not have an extinction peak in the region investigated, therefore Ext/l is taken from 1000 nm.

Spectroscopy

The transparency of the thin films was characterized by UV-vis-NIR spectrophotometry using a pristine glass slide as a background. UV-vis-NIR was conducted from 300 to 1000 nm with an integration time of 1 s (Evolution 201, Thermo Fisher Scientific, USA) and NIR was conducted from 1000-2500 nm (Nicolet iS50R FT-IR, Thermo Fisher Scientific, USA). Transparency at 550 nm was chosen as a standard wavelength to compare between thin film samples. Reflectance measurements were performed in ellipsometry mode (M2000 spectroscopic ellipsometer, J. A. Woollam, USA) with a silicon standard alignment and mirror blank reference using incident light at 750 from perpendicular to the sample (note: 75° was chosen as an arbitrary angle, other angles can be used). Two modes were probed using polarized light (p-polarized light, perpendicular to sample surface and s-polarized light, parallel to sample surface) at 100 revolutions each.

Selective Etching Conditions—Material Synthesis

To synthesize Ti₂C, Ig of Ti₂AlC was added to 10 mL of 10 wt. % HF and stirred for 8 hours at room temperature (RT). To synthesize Ti₃C₂, 1 g of Ti₃AlC₂ was added to 10 mL of 10 wt. % HF and stirred for 10 hours at RT. To synthesize Mo₂C, 1 g of Mo₂Ga₂C was added to 10 mL of 48-50 wt. % HF and stirred for 100 hours at 55° C. To synthesize Mo₂TiC₂, 1 g of Mo₂TiAlC₂ was added to 10 mL 48-50 wt. % HF and stirred for 48 hours at 55° C. To synthesize Mo₂Ti₂C₃, 1 g of Mo₂Ti₂AlC₃ was added to 10 mL of 48-50 wt. % HF and stirred for 96 hours at 55° C. To synthesize Nb₂C and V₂C, 1 g of Nb₂AlC or V₂AlC was added to 10 mL of 48-50 wt. % HF and stirred for 90 hours at RT. To synthesize Nb₄C₃, 1 g of Nb₄AlC₃ was added to 10 mL of 48-50 wt. % HF and stirred for 96 hours at RT. Ta₄C₃ was synthesized from Ta₄AlC₃ by selectively etching Al in 48-50 wt. % HF for 72 hours at RT. All reactions were conducted with stirring set at 400 rpm.

Solution Characterization

MXenes dispersed in deionized water were characterized by zeta (ζ) potential and dynamic light scattering (DLS) measurements (Zetasizer Nano ZS, Malvern Panalytical, UK) and results are summarized in FIG. 18 and Table 1. ζ potential measurements were conducted at neutral pH in a polystyrene folded capillary cell and DLS measurements were conducted using a polystyrene cuvette (concentration ˜0.01 mg/mL). In each case, 5 measurements were recorded and the distributions were averaged in the Malvern software. DLS has been shown to be a reliable method for determining the lateral size of MXene flakes in colloidal solutions.^{[50]} From the particle size intensity distributions, the average lateral sizes range between 100 nm and 500 nm (FIG. 18b), which is expected for flakes that have been subjected to 1 hour of bath sonication.^{[50]}

TABLE 1
Zeta potential (mV) and dynamic light scattering (DLS)
intensity distribution data

| Material | Z-ave (nm) | a_{DLS}(nm) | PDI | ζ (mV) | pH |
|---|---|---|---|---|---|
| Ti₂C | 164.8 | 221.4 | 0.375 | −32.6 | 7.4 |
| Ti₃C₂ | 265.1 | 190.0 | 0.416 | −49.1 | 6.7 |
| Nb₂C | 140.5 | 144.3 | 0.379 | −44.0 | 7.4 |
| Nb₄C₃ | 259.8 | 190.0 | 0.349 | −54.1 | 8.0 |
| V₂C | 154.7 | 190.4 | 0.348 | −32.4 | 7.0 |
| Mo₂C | 208.8 | 277.7 | 0.391 | −44.7 | 7.8 |
| Mo₂TiC₂ | 173.9 | 194.0 | 0.243 | −41.6 | 7.0 |
| Mo₂Ti₂C₃ | 184.1 | 242.1 | 0.255 | −50.1 | 7.8 |
| Ta₄C₃ | 490.8 | 426.1 | 0.511 | −51.0 | 7.4 |

X-Ray Diffraction Analysis

X-ray diffraction (XRD) patterns of the layered Mₙ₊₁Aₓ₊₁Xₙ₊ₓ (MAX) phase precursors and vacuum filtered free-standing films after etching and delamination are presented in FIG. 19. XRD (Rigaku Smartlab) with Cu Kα radiation at a step of 0.020 and a collection time of 0.5s per step was used to determine conversion from precursor to MXene as well as monitor interlayer spacing.

After etching Ti₂AlC with HF/HCl followed by delamination with LiCl, the (002) peak of Ti₂AlC shifts from 13.10 to 8° corresponding to a d-spacing shift from 6.8 to 11.1 Å, resulting from the selective removal of Al, resulting in the formation of surface terminations and intercalated water (FIG. 19a). Changing the etching and processing conditions to HF/TMAOH shifts the (002) of Ti₂AlC to ˜7.2°, indicative of a larger d-spacing of 12.3 Å caused by intercalation of large TMA⁺ ions.^{[29]} Similar trends are observed when reacting Ti₃AlC₂ in similar etchant solutions, however the (002) of Ti₃AlC₂ is located to 9.8° due to the thicker M₃AX₂ structure (FIG. 19b). When reacted with HF/HCl/LiCl or HF/TMAOH, the (002) shifts to 7.3° and 6° (d-spacing of 12.1 Å and 14.7 Å), respectively. In each case, higher ordered Ti₂AlC or Ti₃AlC₂ peaks do not remain in the vacuum filtered film due to the removal of the A layer and preferential orientation of the MXene flakes.

Other M₂AX structures, V₂AlC (and Nb₂AlC) have a (002) peak located at ˜13.5° (12.8°) 20 corresponding to a d-spacing of 6.6 Å (6.9 Å). After etching and intercalation, the V₂C films display a shift in the (002) peak to ˜7.4° with a d-spacing of 11.9 Å (FIG. 19c). Similar to V₂C, Nb₂C shows a (002) peak at 7.0° 20 corresponding to a d-spacing of 12.6 Å (FIG. 19d). Mo₂Ga₂C, a non-MAX layered precursor used to make Mo₂C, exhibits a (002) peak at 9.8° (d-spacing of 9.1 Å) (FIG. 19e).

The first XRD peak for Ti₃AlC₂ is the (002) at ˜9.8°. Similar to other M₃AX₂ structures (like Ti₃AlC₂), Mo₂TiAlC₂ has the (002) peak located ˜9.8° and shifts to a 2θ of ˜5.8° after etching and intercalation with TMA⁺, representing a shift in the d-spacing from 9.1 Å to 15.3 Å (FIG. 19f). A thicker structure, Mo₂Ti₂AlC₃, displays the (004) peak at 15.0° and corresponding MXene free-standing films exhibited the (002) peak shift to 5.1°, indicating a d-spacing of 17.4 Å (FIG. 19g). Other M₄AX₃ structures, Nb₄AlC₃ and Ta₄AlC₃ have the (002) peaks located at a 20 of 7.3° corresponding to a d-spacing of ˜12.1 Å. After etching and intercalation, Nb₄C₃ and Ta₄C₃ films display a shift in the (002) peak to 5.0° with a d-spacing of 17.7 Å (FIG. 19h-i). The c-LP of the free-standing films are included in Table 2.

TABLE 2
X-ray diffraction 2θ (°) of the (002) unless stated
otherwise, d-spacing (Å), and c-LP (Å).

| Material | 2θ (°)(002) | d-spacing (Å) | c-LP (Å) |
|---|---|---|---|
| Ti₂AlC | 13.1 | 6.8 | 13.6 |
| Ti₂C—HF/TMAOH | 7.2 | 12.3 | 24.6 |
| Ti₂C—HF/HCl/LiCl | 8.0 | 11.1 | 22.2 |
| Nb [symbol] AlC | 12.8 | 6.9 | 13.8 |
| Nb [symbol] C—HF/TMAOH | 7.0 | 12.6 | 25.2 |
| V₂AlC | 13.5 | 6.6 | 13.2 |
| V₂C—HF/TMAOH | 7.4 | 11.9 | 23.8 |
| Mo₂Ga₃C | 9.8 | 9.1 | 18.2 |
| Mo₂C—HF/TMAOH | 7.9 | 11.2 | 22.4 |
| Ti₃AlC [symbol] | 9.8 | 9.1 | 18.2 |
| Ti₃C₂—HF/TMAOH | 6.0 | 14.7 | 29.4 |
| Ti₃C₂—HF/HCl/LiCl | 7.3 | 12.1 | 24.2 |
| Mo₂TiAlC₂ | 9.8 | 9.1 | 18.2 |
| Mo₂TiC₂—HF/TMAOH | 5.8 | 15.3 | 30.6 |
| Mo₂Ti₂AlC₃ | 15.0 (004) | 5.9 | 23.6 |
| Mo₂Ti₂C₃—HF/TMAOH | 5.1 | 17.4 | 34.8 |
| Nb₄AlC₃ | 7.3 | 12.1 | 24.2 |
| Nb₄C [symbol] —HF/TMAOH | 5.0 | 17.7 | 35.4 |
| Ta₄AlC [symbol] | 7.3 | 12.0 | 24.0 |
| Ta₄C₃—HF/TMAOH | 5.0 | 17.7 | 35.4 |

[symbol]

indicates data missing or illegible when filed

The Beer Lambert Law

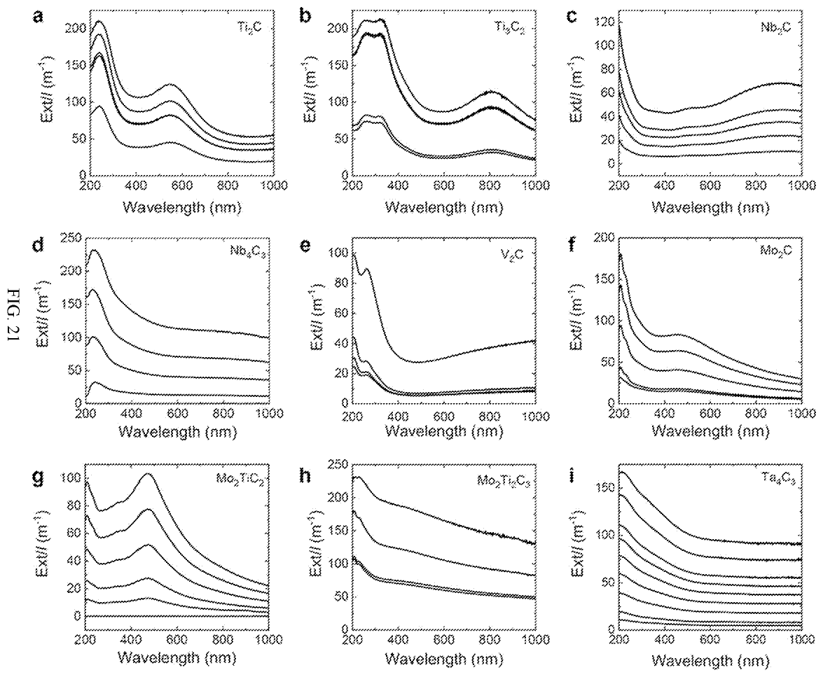

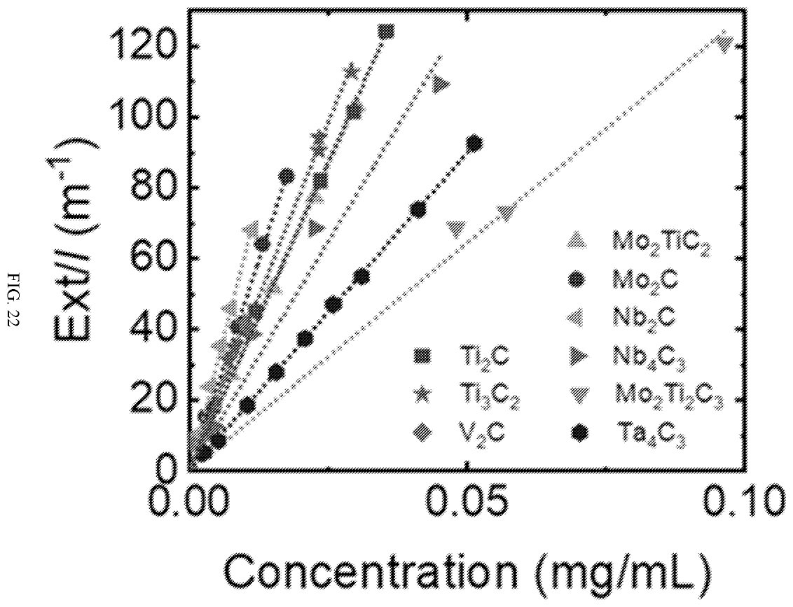

The extinction per path length (Ext/l) was measured across wavelengths from 200 to 1000 nm and the Ext/l at λₘₐₓ was used to build a calibration curve. The extinction divided by path length (Ext/l) for each solution, at the extinction peak maximum in the visible to NIR region (λₘₐₓ), scaled linearly with concentration, following the Beer Lambert law (FIGS. 21, 22).

Line Profile Thickness

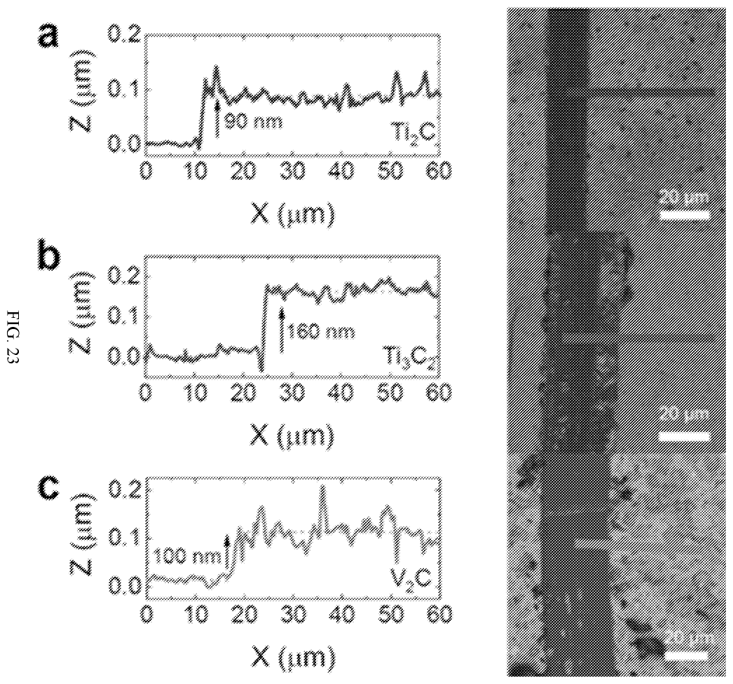

Reflectance measurements were conducted on the thickest Ti₂C, Ti₃C₂, and V₂C films produced via spray coating (FIG. 15a, right-most column) where optical profilometry measurements revealed the thicknesses are approximately 90 nm, 160 nm, and 100 nm, respectively (FIG. 23). Thin film thickness was performed using an optical profilometer (VK-X Series, Keyence, USA) with a 50×objective and the line profile was averaged over 5 lines (0.2 μm step width).

### EMBODIMENTS

The following embodiments are illustrative only and do not serve to limit the scope of the present disclosure or the appended claims.

Embodiment 1. An electrode, comprising: a substrate; a portion of MXene material disposed on the substrate; a hole-injection material disposed on the MXene material; an organic layer in electronic communication with the hole-injection material; and a conductor material in electronic communication with the hole-injection material.

Embodiment 2. The electrode of Embodiment 1, wherein the substrate comprises glass, a polymer, or any combination thereof.

Embodiment 3. The electrode of any one of Embodiments 1-2, wherein the MXene material comprises Ti₃C₂.

It should be understood that other MXene compositions and structures besides Ti₃C₂ can be used, as Ti₃C₂ is simply an illustrative example. As described elsewhere herein, MXenes are two-dimensional (2D) transition-metal carbides, nitrides, or carbonitrides that have the formula Mₙ₊₁Xₙ, where M is an early transition metal (e.g., Ti, V, Nb, Mo), and X is C, N, or both. Thus, MXenes that comprise a transition metal beside Ti can be used.

Embodiment 4. The electrode of any one of Embodiments 1-3, wherein the MXene material is characterized as being in the form of nanosheets. A nanosheet can have a thickness (in the z-direction) of from about 1 to about 100 nm; a nanosheet can be larger than 100 nm in the x- and y-directions, e.g., from 100's of nanometers to even from 1 to 10 micrometers in the x- or y-direction.

Embodiment 5. The electrode of any one of Embodiments 1-4, wherein the hole-injection material is characterized as being chemically neutralized.

Embodiment 6. The electrode of any one of Embodiments 1-5, wherein the hole-injection material is characterized as polymeric.

Embodiment 7. The electrode of Embodiment 6, wherein the hole-injection material comprises a perfluorinated polymer.

Embodiment 8. The electrode of any one of Embodiments 1-7, wherein the hole-injection material comprises one or more of PEDOT, PSS, aniline, and n-GraHIL.

Embodiment 9. The electrode of any one of Embodiments 1-8, wherein the organic layer comprises one or more of TAPC, TCTA, CBP, TPBi, and Ir(ppy)₂acac.

Embodiment 10. The electrode of any one of Embodiments 1-9, wherein the electrode, exclusive of the substrate, is at least partially transparent to visible light. As an example, the electrode, exclusive of the substrate can transmit in the percentage between 10% and 100%; an electrode can be essentially transparent to visible light. An electrode (or a portion thereof) can also, however, exhibit color. Such colors can be, e.g., green; dark purple; golden yellow; grey black; bronze; silver; light blue-silver; dark green-grey; or even silver-gray. The MXene portion of the electrode can be transparent to visible light, but this is not a requirement, as the MXene portion can exhibit color. Such colors can be characterized as, e.g., green; dark purple; golden yellow; grey black; bronze; silver; light blue-silver; dark green-grey; or even silver-gray.

Embodiment 11. The electrode of any one of Embodiments 1-10, wherein the electrode is incorporated into a display device.

Embodiment 12. The electrode of any one of Embodiments 1-11, wherein the conductor material is characterized as metallic.

Embodiment 13. The electrode of any one of Embodiments 1-12, wherein the MXene portion has a work function of from 1.6 eV to 6.25 eV.

Embodiment 14. A method, comprising fabricating an electrode according to any one of Embodiments 1-13.

Embodiment 15. The method of Embodiment 14, wherein the method comprises spin-coating at least one of the portion of MXene material, the hole-injection material, and the organic layer.

Embodiment 16. A method, comprising the use of an electrode according to any one of Embodiments 1-13.

Embodiment 17. A display device comprising an electrode according to any one of Embodiments 1-13.

Embodiment 18. A method, comprising: exposing a plurality of MXene samples to illumination; collecting optical spectra from the plurality of MXene samples; and classifying at least one of the plurality of MXene samples based on an optical spectrum of that at least one of the plurality of MXene samples.

Without being bound to any particular theory or embodiment, each MXene has optical absorption at a specific wavelength. By measuring the absorption spectra, one can distinguish MXenes and determine the quality of the MXene flakes in solution. A non-limiting example is a Ti₃C₂/Ti₂C device in which optical spectra of pure phase MXene are used to detect the dominant material in, e.g., the output spectra and/or plasmon resonance shifts.

Embodiment 19. A photothermal therapy method, comprising: exposing a MXene material sample disposed on or within a subject to near-infrared radiation, the exposing effecting localized heating of a tissue of the subject.

As described elsewhere herein, MXenes can exhibit relatively high extinction in the NIR range (which is also the biological transparency region). In this way, MXene materials can be used in photothermal therapy, e.g., by introducing the MXene material to a subject (e.g., inserting the MXene material beneath the skin of the subject), and exposing the subject (and the MXene material) to NIR illumination, thereby effecting heating of the MXene material and localized heating of subject tissue contacting or nearby to the MXene. Example MXenes that can be used in such applications include MXenes with absorption peaks in the near infrared (e.g., Ti₃C₂) or infrared range of wavelengths (e.g., Nb₂C, Ta₄C₃, and other M₄C₃ MXenes).

Embodiment 20. An electrochromic device, comprising a portion of MXene material; and an electrical current source, the electrical current source in electronic communication with the MXene material, the electrical current source configured to effect application of an electrical current sufficient to effect a change in color, transparency, or both in the MXene material.

Suitable MXene materials are described elsewhere herein. A MXene material can exhibit a baseline color, e.g., dark purple, green, forest green, dark purple, blue, golden yellow, grey-brown, grey black, green-blue, bronze, brown, silver, orange-brown, light blue-silver, grey, dark green-grey, brown, or silver-gray. An electrochromic device can be operated so as to effect a chance in color and/or transparency in the MXene material

Embodiment 21. The electrochromic device of Embodiment 20, wherein the device is configured such that the MXene material modulates passage of illumination to a target region. A target region can be, e.g., a detector, a tissue (e.g., of a subject), and the like. The electrochromic device can be configured as, e.g., a window (such as a so-called “smart window”), an electrochromic mirror, and/or an electrochromic display device. Such an electrochromic device can be operated so as to dynamically tint, e.g., as a smart window on a vehicle that is modulated (via application of current) to exhibit more or less tinting/color as the user may require.

An electrochromic device according to the present disclosure can include one type of MXene material, but can also include a plurality of types of MXene materials. In this way, a device that includes multiple MXene materials can take advantage of the color performance profiles of those materials.

For example, a device that includes a MXene material that can achieve a blue color and a MXene material that can achieve a purple color can be modulated so as to exhibit blue, purple, or even a combination of those colors. Depending on the MXene and its absorption peak (plasmon resonance), the films will have different colors. Thus, different MXenes will have different colors that can cover parts of or even the entire RGB color range. One can use multiple MXenes (as heterostructures or as separate films) in an electrochromic device if one desires to broaden or shrink the wavelengths of interest (e.g., additive or subtractive color). Such MXenes can be present as filters and/or as MXene heterostructures.

Embodiment 22. A sensor, comprising: a MXene portion in electronic communication with a detector configured to detect a signal from the MXene portion, the MXene portion being essentially transparent to visible light.

Embodiment 23. A sensor, comprising: a MXene portion in electronic communication with a detector configured to detect a signal from the MXene portion, the MXene portion being colored.

Embodiment 24. The sensor of Embodiment 23, wherein the color is characterized as green, dark purple, golden yellow, grey black, bronze, silver, light blue-silver, dark green-grey, or silver-gray.

Embodiment 25. The sensor of any one of Embodiments 22-24, wherein the sensor is configured as a plasmon resonance sensor.

1. An electrode, comprising:
a substrate;
a portion of MXene material disposed on the substrate;
a hole-injection material disposed on the MXene material;
an organic layer in electronic communication with the hole-injection material; and
a conductor material in electronic communication with the hole-injection material.

2. The electrode of claim 1, wherein the substrate comprises glass, a polymer, or any combination thereof.

3. The electrode of claim 1, wherein the MXene material comprises Ti₃C₂.

4. The electrode of claim 1, wherein the MXene material is characterized as being in the form of nanosheets.

5. The electrode of claim 1, wherein the hole-injection material is characterized as being chemically neutralized.

6. The electrode of claim 1, wherein the hole-injection material is characterized as polymeric.

7. The electrode of claim 6, wherein the hole-injection material comprises a perfluorinated polymer.

8. The electrode of claim 1, wherein the hole-injection material comprises one or more of PEDOT, PSS, aniline, and n-GraHIL.

9. The electrode of claim 1, wherein the organic layer comprises one or more of TAPC, TCTA, CBP, TPBi, and Ir(ppy)₂acac.

10. The electrode of claim 1, wherein the electrode, exclusive of the substrate, is at least partially transparent to visible light.

11. The electrode of claim 1, wherein the electrode is incorporated into a display device.

12. The electrode of claim 1, wherein the conductor material is characterized as metallic.

13. The electrode of claim 1, wherein the MXene portion has a work function of from 1.6 eV to 6.25 eV.

14. (canceled)

15. (canceled)

16. A method, comprising the use of an electrode according to claim 1.

17. A display device comprising an electrode according to claim 1.

18. (canceled)

19. (canceled)

20. An electrochromic device, comprising a portion of MXene material; and an electrical current source, the electrical current source in electronic communication with the MXene material, the electrical current source configured to effect application of an electrical current sufficient to effect a change in color, transparency, or both in the MXene material.

21. The electrochromic device of claim 20, wherein the device is configured such that the MXene material modulates passage of illumination to a target region.

22. (canceled)

23. A sensor, comprising: a MXene portion in electronic communication with a detector configured to detect a signal from the MXene portion, the MXene portion being colored.

24. The sensor of claim 23, wherein the color is characterized as green, dark purple, golden yellow, grey black, bronze, silver, light blue-silver, dark green-grey, or silver-gray.

25. The sensor of any claim 23, wherein the sensor is configured as a plasmon resonance sensor.
